(19) United States
(12) Plant Patent Application Publication
(10) Pub. No.: US 2002/0062508 P1
(43) Pub. Date: May 23, 2002

(54) Ground Cover rose plant named 'Noalesa'

(76) Inventor: Reinhard Noack, Gutersloh (DE)

Correspondence Address: BURNS DOANE SWECKER & MATHIS L L P, POST OFFICE BOX 1404, ALEXANDRIA, VA, 22313-1404, US

(21) Appl. No.: 09/843,756

(22) Filed: Apr. 30, 2001

(30) Foreign Application Priority Data
Nov. 22, 2000 (DE) ROS 1854

(51) Int. Cl. A01H 5/00

(52) U.S. Cl. PLT/104

(57) ABSTRACT

A new and distinct variety for Ground Cover Rose plant is provided which forms substantially continuously throughout the season attractive long-lasting bright yellow blossoms. The blossoms open on a reliable bases and retain their attractiveness upon aging. The new variety exhibits a compact, spreading and overhanging growth habit that renders it suitable for growing as attractive ornamentation in the landscape. The foilage is glossy and dark green and contrasts well with the bright yellow blossoms. The blossom petals detach cleanly as the blossoms mature. The new variety propagates well by the use of cuttings, and by budding and grafting. Excellent resistance to Blackspot, Powdery Mildew, and Rust is exhibited.

### SUMMARY OF THE INVENTION

Ground Cover rose plants are known and are being used to advantage in an increasing number of landscape plans and home gardens. However, there remains a need for additional varieties of Ground Cover roses with the demand being the greatest for those having highly attractive blossoms in combination with good disease resistance.

The new variety of Rosa hybrida Ground Cover rose plant of the present invention was created at Gütersloh, Germany, by artificial pollination wherein two parents were crossed which previously had been studied in the hope that they would contribute the desired characteristics. The female parent (i.e., the seed parent) was the ‘Korimro’ variety (non-patented in the United States). Such ‘Korimro’ variety possesses light pink to near white single blossoms and is marketed under the IMMENSEE trademark. The male parent (i.e., the pollen parent) was an unnamed seedling maintained by Noack Rosen at Gütersloh, Germany (non-patented in the United States). The parentage of the new variety can be summarized as follows:

### ‘Korimro’×Unnamed Seedling

The seeds resulting from the above pollination were sown and small plants were obtained which physically and biologically differ from each other. Selective study resulted in the identification of a single plant to the new variety.

It is found that the new variety of Ground Cover rose plant possesses the following combination of characteristics:

(a) forms attractive bright yellow blossoms,

(b) young plants exhibit an upright growth habit that becomes spreading, dense, and compact with maturity,

(c) forms attractive dark green glossy foliage that contrasts well with the bright yellow blossoms,

(d) exhibits excellent disease resistance with respect to Blackspot, Powdery Mildew, and Rust, and

(e) is particularly well suited for growing as attractive ornamentation in the landscape.

The new variety meets the needs of the horticultural industry and can be grown to advantage as attractive ornamentation in parks, gardens, public areas, and residential landscapes.

The new variety of the present invention beginning in 1994 has been asexually reproduced by the rooting of cuttings, by budding, and by grafting. Such asexual reproduction as performed at Gütersloh, Germany, has demonstrated that the characteristics of the new variety are firmly fixed and stable and are strictly transmissible from one generation to another.

The new variety has been named the ‘Noalesa’, and recently has been marketed under the JACOBS KRÖNUNG trademark.

### BRIEF DESCRIPTION OF THE PHOTOGRAPHS

The accompanying photographs show as nearly true as it is reasonably possible to make the same, in color illustrations of this character, typical specimens of the new variety while growing in the landscape. The illustrated rose plants were photographed during July, 1996, while growing in the field at Gütersloh, Germany. Such plants were budded approximately one and one-half years earlier.

FIG. 1 illustrates the overall appearance of typical plants of the new variety while bearing a profusion of blossoms in various stages of opening.

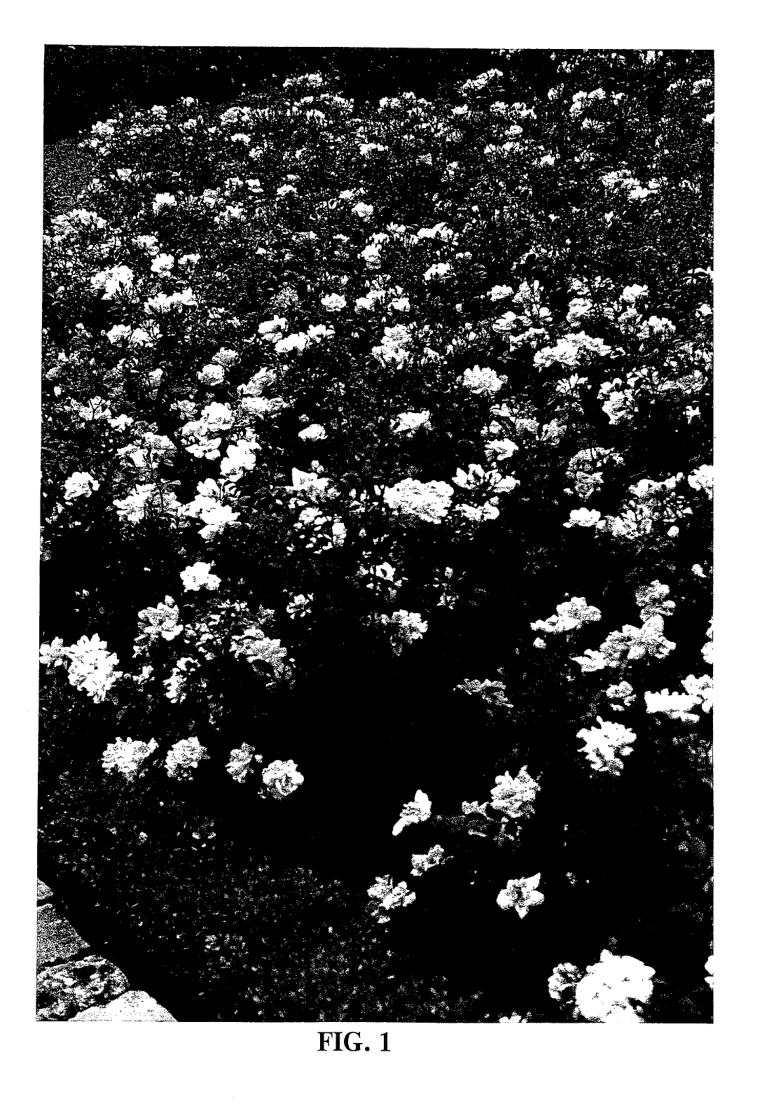

FIG. 2 illustrates a closer view of typical blossoms, buds, and foliage of the new variety wherein the bright yellow blossoms are shown to contrast nicely with the glossy dark green foliage.

### DETAILED DESCRIPTION

The chart used in the identification of the colors is that of The Royal Horticultural Society (R.H.S. Colour Chart) of London, England. Color terminology in common terms sometimes is included as an aid to the reader. Such terminology is to be accorded its customary dictionary significance. The description is based on the observation of specimens of the new variety while growing outdoors during July at Gütersloh, Germany.

Class: Ground cover:

Parentage:

Female.—‘Korimro’ (non-patented in the United States).

Male.—Unnamed Seedling maintained by Noack Rosen at Gütersloh, Germany (non-patented in the United States).

Plant:

Form.—Vigorous, young plants exhibit an upright growth habit, then as the plant matures they spread to form a compact bush that serves well as ground cover with dense foliage.

Branches: Color:

Young stems.—Green and slightly reddish, and with a smooth surface.

Adult wood.—Medium green and a little darker than the young stems, and with a smooth surface.

Thorns:

Size.— Medium, approximately 5 mm in length on average, and slightly curved downward, and concave on the under surface.

Position.—Irregular.

Color.—Initially light reddish-brown and changing to dark brown with maturity. quantity: moderate and typical for a Ground Cover rose plant.

Leaves:

Stipules.—Light green in coloration.

Leaflets.—Number: commonly 5 and 7.

Configuration.—Elliptical, concave and pointed.

Quantity.—Very abundant.

Size.—Small to medium.

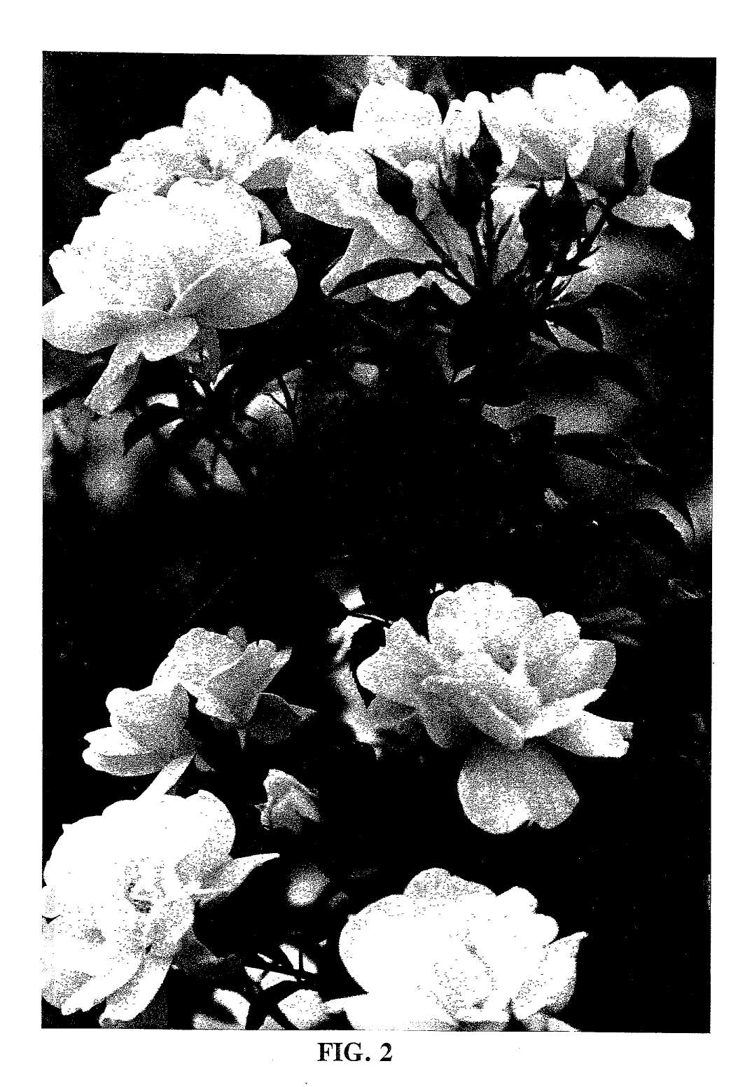

Serration—Slightly serrate, as illustrated in FIG. 2.

Color (young foilage):

Upper surface.—Light green.

Under surface.—Lighter green than the upper surface.

Color (adult foilage):

Upper surface.—Dark green.

Under surface.—Somewhat darker green than new foilage.

General appearance.—Dense, dark green foilage that is glossy on the upper surface and matte on the under surface.

Texture.—Leathery.

Stipules.—Light green in coloration.

Inflorescence

Number of flowers.—In clusters as a large spray.

Peduncle.—Light green in coloration.

Sepals:

Shape.—Pointed as illustrated in FIG. 2.

Buds:

Length.—Approximately 3 cm on average.

Color when opening.—Mimosa Yellow, Yellow Group 8A to 8C.

Flower:

Form.—Initially cup-shaped with flattening upon maturity.

Appearance.—Smooth and somewhat satiny on both petal surfaces.

Diameter.—5 cm on average.

Color (when opening begins):

Upper surface.—Mimosa Yellow, predominantly Yellow Group 8A.

Under surface.—Mimosa Yellow, predominantly Yellow Group 8A to 8C.

Color (when blooming):

Upper surface.—Mimosa Yellow, predominantly Yellow Group 8B. The yellow coloration of pollen and reproductive organs commonly is also visible.

Under surface.—Mimosa Yellow, predominantly Yellow Group 8A to 8C.

Color (at the end of opening):

Upper surface.—Mimosa Yellow, Yellow Group 8A to 8C.

Under surface.—Mimosa Yellow, Yellow Group 8A to 8C.

Fragrance.13 Slight.

Lasting quality.—Very good.

Petal form.—Broad, fan-shaped, curved with a lightly indented central area and margin areas that are slightly curved towards the center.

Petaloids.—Some are formed.

Petal arrangement.—Generally arranged in a regular pattern with overlapping edges.

Petal drop.—Good with the petals detaching cleanly.

Stamens.—Regularly arranged around the style.

Filaments.—Regularly arranged around the style and are nearly transparent in appearance.

Pollen.—Light yellow in coloration.

Stigmas.—Light yellow in coloration.

Styles.—Thin, regularly arranged at the center, and tend to be of substantially the same length.

Hip.—Cup-shaped, and upon maturity change from green to red in coloration.

Development:

Vegetation.—Strong and vigorous.

Blooming.—Abundant in sprays.

Aptitude to bear fruit.—Slight.

Winter hardiness.—Good and comparable to that of the ‘Flower Carpet Pink’ variety (U.S. Plant Pat. No. 7,282).

Resistance to diseases.—Excellent with respect to Blackspot, Powdery Mildew, and Rust.

I claim:

1. A new and distinct variety of Ground Cover Rose plant characterized by the following combination of characteristics:
(a) forms attractive bright yellow blossoms,
(b) young plants exhibit an upright growth habit that becomes spreading, dense, and compact with maturity,
(c) forms attractive dark green glossy foilage that contrasts well with the bright yellow blossoms,
(d) exhibits excellent disease resistance with respect to Blackspot, Powdery Mildew, and Rust, and
(e) is particularly well suited for growing as attractive ornamentation in the landscape;
substantially as herein shown and described.
